(19) United States
(12) Patent Application Publication
(10) Pub. No.: US 2008/0033536 A1
(43) Pub. Date: Feb. 7, 2008

(54) STABILITY OF BIODEGRADABLE METALLIC STENTS, METHODS AND USES

(75) Inventor: Eric Wittchow, Nuernberg (DE)

Correspondence Address: POWELL GOLDSTEIN LLP, ONE ATLANTIC CENTER, FOURTEENTH FLOOR 1201 WEST PEACHTREE STREET NW, ATLANTA, GA, 30309-3488, US

(73) Assignee: BIOTRONIK VI PATENT AG, Baar (CH)

(21) Appl. No.: 11/835,018

(22) Filed: Aug. 7, 2007

(30) Foreign Application Priority Data
Aug. 7, 2006 (DE) 10 2006 038 235.8

(51) Int. Cl. A61F 2/06

(52) U.S. Cl. 623/1.38; 623/1.46

(57) ABSTRACT

A biodegradable metallic stent having improved stability properties after implantation of the stent, methods for producing such a stabilized stent, methods for improving the stabilization of a biodegradable metallic stent, and a method for improving the stability of a biodegradable metallic stent by using vasodilator active ingredients.

### PRIORITY CLAIM

This patent application claims priority to German Patent Application No. 10 2006 038 235.8, filed Aug. 7, 2006, the disclosure of which is incorporated herein by reference in its entirety.

### FIELD

The present disclosure relates to a biodegradable metallic stent having improved stability properties after implantation of the stent, a method for producing such a stabilized stent, and a method for improving the stabilization of a biodegradable metallic stent.

### BACKGROUND

Stents are generally endovascular prostheses and/or implants which are used for treating stenoses, for example. Stents are additionally known for the treatment of aneurysms.

Stents basically have a support structure which is capable of supporting the wall of a vessel to widen the vessel and/or bypass an aneurysm. For this purpose, stents are inserted in a compressed state into the vessel and then expanded and pressed against the vascular wall at the location to be treated. This expansion may be performed with the aid of a balloon catheter, for example. Alternatively, self-expanding stents are also known. Self-expanding stents are constructed from a super-elastic metal, such as nitinol, for example.

Stents are currently divided into two basic types, permanent stents and biodegradable stents. Permanent stents are implanted in such a way that they may remain in the vessel for an undetermined period of time. Biodegradable stents, in contrast, are degraded over a predetermined period of time in a vessel. Preferably, biodegradable stents are first degraded when the traumatized tissue of the vessel has healed and thus the stent no longer has to remain in the vascular lumen.

For example, biodegradable metal alloys, polymers, or composite materials which have a sufficient structural carrying capacity to be able to support the vascular lumen over a predetermined period of time are known as biodegradable stent materials.

It is known from International Patent Publication No. WO 2005/102222 that metallic stents may possibly cause irritations of the vascular tissue surrounding the stents because the metal is typically much harder and more rigid than the vascular tissue surrounding it. Therefore, damage to the vascular tissue may occur or undesired biological reactions of the tissue may be induced.

In order that the expansion of the vessels by stent implants, so-called stenting, is successful, a stent is selected so that vascular constrictions do not occur again in the area of the inserted stent.

With biodegradable metallic stents, a significant diameter loss of the stent has been observed in animal experiments, in particular, within the first two weeks after implantation. The success of the stenting is thus also a function of a biodegradable metallic stent not having a significant diameter loss after implantation.

### SUMMARY

The present disclosure provides several exemplary embodiments of the present invention.

One aspect of the present disclosure provides a biodegradable metallic stent, comprising (a) a coating comprising one or more vasodilator active ingredients selected from the group consisting of calcium channel blockers, nitrovasodilators, rho-kinase inhibitors, endothelin receptor antagonists, serotonin antagonists, adrenoreceptor antagonists, potassium channel openers, angiotensin conversion enzyme (ACE) inhibitors, musculotropic and neurotropic-musculotropic spasmolytics, and vasodilators having unknown action mechanisms, the active ingredient or the active ingredients of which regulate down spasmogenically active messenger agents, phosphodiesterase-5 (PDE-5) inhibitors, TRP inhibitors or activators, the active ingredients which activate or inhibit TRP channels and (b) a matrix associated with the coating such that the vasodilator active ingredient has a vasodilator effect in the area of the stent implantation contemporarily to the biodegradation of the stent after implantation.

Another aspect of the present disclosure provides a method for improving the stability of a biodegradable metallic stent, comprising the steps of (a) providing a biodegradable metallic stent; (b) providing one or more vasodilator active ingredients selected from the group consisting of calcium channel blockers, nitrovasodilators, rho-kinase inhibitors, endothelin receptor antagonists, serotonin antagonists, adrenoreceptor antagonists, potassium channel openers, angiotensin conversion enzyme (ACE) inhibitors, musculotropic and neurotropic-musculotropic spasmolytics, and vasodilators having unknown action mechanisms, the active ingredients of which regulate down spasmogenically active messenger agents, phosphodiesterase-5 (PDE-5) inhibitors, TRP inhibitors or activators, the active ingredients which activate or inhibit TRP channels; and (c) coating the biodegradable metallic stent with the vasodilator active ingredient in a suitable matrix, wherein the biodegradable metallic stent is coated so that the vasodilator active ingredient has a vasodilator effect in the area of the stent implantation contemporarily to the biodegradation of the stent after implantation.

A further aspect of the present disclosure provides a method for producing a biodegradable metallic stent, comprising (a) providing a biodegradable metallic stent, (b) providing one or more vasodilator active ingredients selected from the group consisting of calcium channel blockers, nitrates, rho-kinase inhibitors, endothelin receptor antagonists, serotonin antagonists, adrenoreceptor antagonists, potassium channel openers, angiotensin conversion enzyme (ACE) inhibitors, musculotropic and neurotropic-musculotropic spasmolytics, and vasodilators having unknown action mechanisms, the active ingredients of which regulate down spasmogenically active messenger agents, phosphodiesterase-5 (PDE-5) inhibitors, TRP inhibitors or activators, and the active ingredients which activate or inhibit TRP channels; and (c) coating the biodegradable metallic stent with the one or more vasodilator active ingredient in a suitable matrix, wherein the biodegradable metallic stent is coated in such a way that the vasodilator active ingredient has a vasodilator effect in the area of the stent implantation contemporarily to the biodegradation of the stent after implantation.

An additional aspect of the present disclosure provides a method for improving the stability of a biodegradable metallic stent, comprising coating a biodegradable metallic stent with one or more vasodilator active ingredients in a suitable matrix such that the one or more vasodilator active ingredients have a vasodilator effect in the area of the stent implantation contemporarily to the biodegradation of the stent after implantation.

For purposes of the present disclosure, contemporarily means that the vasodilator active ingredient(s) have a vasodilator effect in the area of the stent implantation in a time span from a short time before up to a short time after the beginning of the degradation of the implanted stent and continues the vasodilator effect until the biodegradation of the stent is terminated, preferably until the concentration of hydroxide ions released by the biodegradation of the stent is no longer sufficient to trigger and/or mediate a vascular spasm, also preferably within four months after stent implantation, more preferably within one to eight weeks, especially preferably within one to three weeks after stent implantation.

The present invention is based in part on the new finding of the inventors that the reason for the significant diameter loss of biodegradable metallic stents after implantation is represented by a vascular spasm, in particular, a permanent vascular spasm in the area of the implanted stent. Such a vascular spasm begins between one and three weeks after implantation. In contrast, such a phenomenon was not observed after implantation of biodegradable polymer stents or permanent metallic stents.

Building in part on this new finding, the inventors have also found that a possible cause for the vascular spasm is the metal ions which are released and/or the hydroxide ions which form in the area of the implanted biodegradable metallic stent upon the biodegradation of the metallic stent.

One possible explanation for this is that after implantation, metal and hydroxide ions are present in such a concentration in the vascular area of the stent, released by the biodegradation of the metallic stent, that the concentration of metal and hydroxide ions, above all of hydroxide ions, is sufficient to open the calcium ion channels of the vascular tissue, probably the L-type calcium ion channels, in the area of the stent and thus cause an inflow of calcium ions from the extracellular space into the smooth vascular muscle cells. It is suspected that the actual reactions which mediate such a vascular spasm are triggered by the rise of the intracellular calcium on concentration in the vascular muscle cells.

According to the newest findings, it has been shown that, in addition to the calcium channels just cited, the so-called TRP channels (Transient Receptor Potential) play a not entirely insignificant role in the proliferation, intima thickening, angiogenesis, and the formation of a vascular spasm. The TRP-C1, TRP-C3, TRP-C4, and TRP-C6 channels are in consideration above all, because these are especially influenced by hydroxide ions and some ion metal ions.

This explanatory theory is supported by findings from other technical fields, namely that the pH value may have an influence on the contractility of blood vessels. This finding was used in the 1980s to trigger spasms of this type in symptomatic patients (see, Weber S: “Systemic alkalosis as a provocative test for coronary artery spasm in patients with infrequent resting chest pain”; Am Heart J. 1988, 115:54-9). The regulatory processes on which this is based are described in textbooks of pharmacology (e.g., Mutschler et al.; “Arzneimittelwirkungen: Lehrbuch der Pharmakologie und Toxikologie [Pharmaceutical Effects: Textbook of Pharmacology and Toxicology]”). The general finding is that hydroxide ions have an influence on the L-type calcium ion channels of smooth vascular muscle cells and may open the L-type calcium ion channels, thereby allowing an inflow of calcium ions to occur from the extracellular space into the smooth vascular muscle cells. Furthermore, it is described that the concentration of free calcium ions in a resting vascular muscle cell is only approximately 1:10,000 in comparison to the extracellular space and the intracellular calcium ion concentration suddenly rises to approximately 1:1000 due to the external stimulus of the hydroxide ions. The calcium ions are bound to calcium-binding proteins (inter alia, calmodulin) intracellularly and the actual reactions, namely the mediation of a vascular spasm, are triggered by proteins in the vascular muscle cell activated in this way.

Groschner et al. (“Intracellular pH as a Determinant of Vascular Smooth Muscle Function”, J Vasc Res 2006; 43:238-250) concern themselves with the intracellular pH value increase and its direct effect on the contractility, growth, and proliferation of smooth vascular muscle cells. In regard to the contractility, it has been established that no generally valid statement may be made for an intracellular pH value regulation, but rather there is a complex interweaving of relationships between intracellular pH value and vascular tonus. Furthermore, it is described that the effects of an intracellular pH value increase for different vessels appear to be strongly dependent on the type of the vessel and also on experimental conditions. In large arteries, the resting tonus appears to be strongly coupled to the pH value, an alkalosis triggering a vasoconstriction. In contrast to this, small arteries and arterioles appear to be less sensitive to pH value oscillations.

In vitro tests have also shown that vascular spasms of this type may develop a force of up to 2.5 bar.

However, it has not heretofore been disclosed that biodegradable metallic stents have a significant diameter loss in the area of the stent implantation after implantation, that the significant diameter reduction is mediated by a vascular spasm, and that probably this vascular spasm is (partially) triggered and/or mediated by released metal hydroxide ions from the biodegradable stent.

Therefore, the art does not anticipate the findings of the inventors described above relating to the cause of the observed vascular constrictions, namely, the vascular spasm, and the cause of such vascular spasm, namely, the elevated concentration of metal hydroxide ions as a result of the stent degradation, but rather represents a possible explanation which suggests itself in retrospect in consideration of the findings of the inventors.

Accordingly, it has surprisingly advantageously been shown that the reactions (partially) triggered and/or mediated via the released metal hydroxide ions may be prevented and/or reduced by the release of the metal hydroxide ions by degradation of the metallic stent and the contemporary vasodilator effect in the area of the stent implantation of one or more vasodilator active ingredients.

As a result, the vasodilator effect of the active ingredient(s) contemporarily to the formation and release of the metal hydroxide ions from a biodegradable metallic stent may prevent and/or reduce a significant diameter loss of the biodegradable metallic stent.

A biodegradable metallic stent according to one aspect of the present disclosure is preferably cylindrical and expandable.

Vessels which are suitable for such stent implantations are human or animal blood vessels, in particular, arteries and veins; of these, arteries are particularly preferred. Stent implantations in large arteries are particularly preferred.

For purposes of the present disclosure, vascular lumen means the cavity of a blood vessel.

For purposes of the present disclosure, elution or eluting of the active ingredient(s) means that the active ingredient(s) is released from the carrier matrix.

The feedback loop pH value/ion channels/calcium economy/vascular contraction may be interrupted at various points by suitable vasodilator active ingredients.

Suitable vasodilator active ingredients which are released directly from the coated stent to the vascular lumen, preferably the vascular lumen surrounding the stent and adjoining areas, more preferably the vascular wall adjoining the stent, are selected from the group consisting of calcium channel blockers, nitrovasodilators, rho-kinase inhibitors, endothelin receptor antagonists, serotonin antagonists, adrenoreceptor antagonists, potassium channel openers, angiotensin conversion enzyme (ACE) inhibitors, musculotropic and/or neurotropic-musculotropic spasmolytics, vasodilators having unknown action mechanisms (e.g., hydrazine derivatives, cicletanine), of the active ingredient(s) which regulate down spasmogenically active messenger agents, such as endothelin, thromboxane, serotonin, 5-HT, ADP (vasopressin), PAF (platelet activating factor), and the like, in particular regulate them down by gene therapy, the phosphodiesterase-5 (PDE-5) inhibitors, such as, but not limited to, sildenafil (Viagra), tadalafil (Cialis), or vardenafil (Levitra, Vivanzia), TRP inhibitors or activators, as well as the active ingredient(s) which may activate or inhibit TRP channels.

### Calcium Channel Blockers

Calcium channel blockers prevent the direct inflow of calcium ions into the vascular muscle cells. It has been shown in numerous in vitro experiments that pH-induced spasms may be largely avoided in this way. Calcium channel blockers having an effect on the L-channels are preferred.

Suitable calcium channel blockers are, for example,
dihydropyridines (substances of the nifedipine type):
   nifedipine, nisoldipine, nicardipine, nitrendipine, nimodipine, felodipine, isradipine, nilvadipine, amlodipine, lercanidipine, lacidipine, and the like.
phenylalkylamines (substances of the verampamil type):
   verampamil, gallopamil, and the like.
benzothiazepines (substances of the diltiazem type):
   diltiazem, and the like.
others:
   fendiline

Lercanidipine, lacidipine, amlodipine, and nitrendipine are preferred.

### Nitrovasodilators

Organic nitrates, nitrites, and amino acids which are subject to a metabolic conversion into NO (the actual active substance) are referred to as nitrovasodilators. Therefore, nitrates are typically prodrugs. NO stimulates the cytosolic guanylate cyclase, which catalyzes the formation of cyclic guanosine monophosphate (cGMP) from guanosine triphosphate (GTP). CGMP, in turn, causes the reduction of the intracellular calcium ion concentration and thus a reduction of the vascular tonus.

Suitable active ingredients are, for example:

Glyceroltrinitrate (nitroglycerin), isosorbide dinitrate, isosorbide-5-mononitrate, pentaerythrityl tetranitrate, molsidomine, linsidomine, nicorandil, nitroprusside sodium, the amino acid L-arginine and polypeptides which entirely or partially consist of L-arginine, and the like.

### Rho-Kinase Inhibitors

Rho-kinase belongs to the family of serine/threonine kinases and is activated by various vasoactive mediators, such as catecholamine, UII, thromboxane, and serotonin. Rho-kinase plays a key role in the vascular contraction of the smooth muscle. Rho-kinase-induced contraction may be induced in all vascular beds of the various animal species examined (rats, mice, rabbits, pigs) and may be inhibited as a function of concentration by selective rho-kinase inhibitors (Steioff, Kerstin, “Multiple Wirkungen der Rho-kinas: Neue Möglichkeiten zur Therapie von Bluthochdruck [Multiple Effects of Rho-kinase: New Possibilities for the Treatment of High Blood Pressure]”), doctoral thesis 2005, Frankfurt am Main, http://publikationen.ub.uni-frankfurt.de/volltexte/2005/1579/pdf/SteioffKerstin.pdf).

Suitable rho-kinase inhibitors are, for example:

Fasudil (HA 1077), fasudil derivative (HA 1152), and Y 27632, and the like. (particularly described in Hu et al, “Rho-kinase inhibitors as potential therapeutic agents for cardiovascular diseases”, Cur. Opin. Investig. Drugs 2003; 4 (9):1065-1075).

### Musculotropic or Neurotropic-Musculotropic Spasmolytics

Independently of the vegetative enervation, the smooth musculature may be awoken by direct action of suitable active ingredients on the smooth muscle cells. Active ingredients acting in this way are referred to as musculotropic or papaverine-like spasmolytics.

Suitable musculotropic spasmolytics are, for example:
Papaverine, tiropramide, and the like.

Active ingredients which have both neurotropic (parasympatholytic) and also musculotropic spasmolytic properties occupy an intermediate position between the parasympatholytics and the musculotropic spasmolytics.

Suitable neurotropic-musculotropic spasmolytics are, for example:
drofenine, mebeverine, oxybutynine, propiverine, and the like.

### Endothelin Receptor Antagonists

Endothelins (ET-1, ET-2, and ET-3) stimulate the ETA receptors on the smooth vascular musculature and result in a vascular constriction. The vasoconstrictive property of ET-1 is approximately 10 times greater than that of angiotensin II. Endothelial cells react to damage (e.g., due to high pH values) by excreting endothelins. Endothelin receptor antagonists block the ETA and the ETB receptors and thus prevent excess vascular contraction.

A suitable endothelin receptor antagonist for this purpose is, for example, bosentane.

The most recent findings have shown that selective blocking of ETA receptors also offers advantages.

### Potassium Channel Openers:

The opening probability of calcium channels in the cell membrane (e.g., in the smooth vascular musculature of arterial blood vessels) determines the level of the resting potential. With rising opening probability, the resting membrane potential is displaced in the direction of the potassium equilibrium potential, and the membrane hyperpolarizes. As a result, the calcium inflow through voltage-dependent calcium channels drops, which results in a reduction of the intracellular calcium and thus in vasodilation.

Suitable potassium channel openers are, for example, diazoxide, minoxidil, nicorandil, pinacidil, levcromakalim and the like.

### Vasodilators Having Unknown Action Mechanisms

The action mechanisms of the hydrazine derivatives is unknown up to this point. The essential hemodynamic effect comprises a reduction of the peripheral vascular resistance. The action mechanism of cicletanin is also unknown. It has a vasodilator effect, inter alia, probably by stimulation of the muscarine receptors on endothelial cells and by inhibition of voltage-dependent calcium channels in the vascular musculature.

Suitable substances are, for example, hydralazine, dihydralazine, and cicletanin.

### Serotonin Antagonists

Substances which antagonize the action of the spasmogen serotonin are referred to as serotonin antagonists. These substances frequently also have an influence on the calcium channels in the intracellular calcium store.

Suitable substances are, for example, cinnarizine and flunarizine.

### Angiotensin Conversion Enzyme (ACE) Inhibitors

Suitable active ingredients are, for example:
benazepril, captopril, cilazapril, enalapril, fosinopril, imidapril, lisinopril, moexipril, perindopril, quinapril, ramipril, spirapril, trandolapril, and the like.

### Adrenoreceptor Antagonists

Suitable active ingredients are, for example:
doxazosin, prazosin, uradipil, sotalol, propanolol, pindolol, carvedilol, metoprolol, atenolol, talinolol, bisoprolol, nebivolol, betaxolol, and the like.

### PDE-5 Inhibitors

Suitable active ingredients are, for example:
sildenafil (Viagra), tadalafil (Cialis), vardenafil (Levitra, Vivanzia)

Suitable active ingredients which are discharged directly from the coated stent to the vascular lumen, preferably to the vascular lumen enclosing the stent and adjoining areas, more preferably to the vascular wall enclosing and adjoining the stent, have the following properties in preferred exemplary embodiments:

pH Value Stability

Because the increase of the pH value which directly triggers local vascular spasms may also result in chemical changes, in particular, in deprotonation of the suitable active ingredient, a suitable active ingredient is in its pharmaceutically active form at pH less than or equal to 11, more preferably pH less than or equal to 12, and/or the chemical conversion occurs sufficiently slowly in this pH range.

### Lipophilicity

A preferred active ingredient is also lipophilic, because lipophilic active ingredients may use the vascular wall as a store and may, therefore, remain longer in the lipophilic vascular compartments which enclose the stent and in the adjoining areas thereof, and may thus result in permanent dilation, even if the primary active ingredient discharge by the stent is already largely finished (depot effect).

Thus, a stent eluting sirolimus which discharges its active ingredient within 7 days displays the same activity in human experiments over 6 and 24 months as a stent in which sirolimus is discharged over a time of 30 days (Sousa et al., “Lack of neointimal proliferation after implantation of sirolismus-coated stents in human coronary arteries: a quantitative coronary angiography and three-dimensional intravascular ultrasound study”, Circulation, 2001; 103:192-195; Sousa et al., “Two-year angiographic and intravascular ultrasound follow-up after implantation of sirolismus-eluting stent in human coronary arteries”, Circulation, 2003, 107: 381-383).

In particular, lipophilic calcium channel blockers selected from the following group are preferred: lercanidipine, lacidipine, amlodipine, and nitrendipine.

### No Tolerance Development

When a suitable active ingredient is used, preferably no tolerance development is to occur.

Glycerin trinitrate is an example of an active ingredient which has such a tolerance, a so-called nitrate tolerance. After approximately 24 hours, the activity of glycerin nitrate already decreases, the processes which are responsible for this not yet having been completely explained (Munzel et al., “Explaining the phenomenon of nitrate tolerance”, Circ Res., 2005 Sep. 30, 97(7): 618-628).

In particular, nitrates selected from the following group are preferred: pentaerythryl tetranitrate, molsidomine, and linsidomine, because these substances release NO non-enzymatically, which very probably represents the cause of the nonexistent tolerance development.

A further active ingredient which also has no tolerance development is L-arginine. Although this substance is not a nitrate, it also releases NO.

### High Activity

Active ingredients which have a high activity are preferred, so that a less active ingredient is needed for coating the stent to maintain an active dose for as long as possible.

In this way, a preferred active ingredient of this type may result in the layer thickness of the coated stent not being as thick as layer thicknesses of less-active active ingredients.

### Long Half-Life

Preferred active ingredients additionally have a long half-life.

An active ingredient preferred in this way may result in the layer thickness of the coated stent not being as thick as the layer thickness of active ingredients having a shorter half-life.

The preferred exemplary embodiments are additionally described in the subclaims.

In a preferred exemplary embodiment, the elution time of the vasodilator active ingredient(s) from the matrix [TE(W)] corresponds to the degradation time of the stent according to one aspect of the present disclosure [TD(S)].

This exemplary embodiment of such a degradation or elution behavior may be implemented by partially coating the stent with the matrix. An example of a stent coated in this way is disclosed in International Patent Publication No. WO 2005/102222.

The vasodilator active ingredient(s) preferably elute over a period of time of 4 months, more preferably over a period of time of at least 3 to 8 weeks beginning with the stent implantation.

In a further preferred exemplary embodiment, the elution time of the active ingredient(s) from the matrix [TE(W)] corresponds to the degradation time of the active ingredient depot of the matrix on the stent according to the present disclosure [TD(D)] and [TD(D)] and [TE(W)] are each less than the degradation time of the stent [TD(S)] and [TD(S)] is less than or equal to the elution time of the active ingredient(s) from the vascular wall [TE(G)].

This exemplary embodiment of such a degradation or elution behavior may be implemented by the classic stent coating. In this case, there is complete coating of the stent both on the interior, which faces toward the vascular lumen, and on the mural side, the exterior side, which faces toward the vascular wall. The matrix thus represents an active ingredient depot. This exemplary embodiment is preferably suitable for one or more lipophilic vasodilator active ingredients which elute from the matrix and are enriched in such a way in the cells of the vascular wall which enclose the stent that an active ingredient depot arises there. The vasodilator active ingredient(s) then elute from the active ingredient depot of the vascular wall contemporarily with the biodegradation of the stent and thus with the release of hydroxide ions from the stent.

In a further preferred exemplary embodiment, the degradation time of the stent [TD(S)] is less than the elution time of the active ingredient(s) from the vascular wall [TE(G)] or less than the elution time of the active ingredient(s) from the matrix of the stent [TE(W)], [TE(G)] corresponding to [TE(W)].

This exemplary embodiment of such a degradation and/or elution behavior may be implemented by a “detachable” coating of the matrix of the stent.

A coating of a stent which is “detachable” in this way is distinguished, in particular, that the matrix is applied to a coating area of the surface of the main body provided for this purpose in such a way that
the coating area is divided into an uncoated partial area and a partial area coated with the matrix, the coated partial area covering 5 to 80% of the surface of the stent;
a distance of an arbitrary point of the surface in the coated partial area to the closest uncoated partial area is less than 60 μm; and
a distance of an arbitrary first boundary point of the surface in the coated partial area to a second boundary point in the same coated partial area which is furthest away from the first boundary point is at most 400 μm.

Through such a coating, it is possible to disentangle the degradation behaviors of the matrix and the stent, and thus to tailor the elution of the active ingredients and the procedures during the degradation more precisely and to restrict possibly required modifications to only a part of the system. Because of the main body degradation, the coated partial areas will detach from the surface of the main body and, if in contact with the tissue, grow into the surrounding tissue. The coated partial areas function in the surrounding tissue as local active ingredient depots which are not in contact with the main body of the implant locally or in regard to the elution and degradation processes.

In the present preferred exemplary embodiments, if two, three, or more vasodilator active ingredients are incorporated in the matrix, the vasodilator active ingredients may be eluted independently of one another.

In a further preferred exemplary embodiment, the matrix of a stent additionally comprises one or more active ingredients from the group consisting of anti-inflammatory and/or antiproliferative active ingredients and/or antisense nucleotides and/or biphosphonates and/or antibodies and/or progenitor cells, and the like.

For purposes of the present disclosure, a suitable matrix may thus contain or enclose the active ingredient(s), for example, in foams, in channel systems, and/or in layer systems, and/or may particularly control the release of the active ingredient(s). For purposes of the present disclosure, a matrix may additionally support the structure of the stent, and offer structural integrity and/or structural barriers. The release of the active ingredients from a suitable matrix may be controlled via the matrix material or the type of coating, as already described in the art.

For purposes of the present disclosure, a suitable matrix may be selected from the group consisting of
polymers, such as polylactides (PLA poly lactide acid), methylmethacrylate (MMA), polyhydroxy butyric acid (PHB poly hydroxy butyrate), poly(orthoesters) (POE), polypeptides, polysaccharides, phosphorylcholine (PC), hyaluronic acid (HA), cholesterol and the like, or
fats, such as natural or modified soybean oil, and the like.

Preferred matrix materials are bioresorbable. The suitable matrix materials are preferably not degraded before the complete degradation of the metallic stent.

The quantity of the matrix coating is dependent on the activity of the vasodilator active ingredients. The active ingredient quantity on a stent of 10 mm length and a diameter in the dilated state of approximately 3 mm is preferably in the range from 1 to 500 μg, more preferably in the range from 10 to 200 μg. In general, the stent may have a length of 8 to 80 mm and a diameter of 2 to 12 mm, depending on the area of application. The active ingredient quantity is adapted depending on the length and diameter.

For purposes of the present disclosure, a coating is an envelopment of the biodegradable metallic stent and/or a filling and/or a charging of openings, preferably holes and/or cavities and/or cages in and/or on the stent with a suitable matrix, which contains one or more vasodilator active ingredients and possibly one or more further active ingredients, the biodegradable metallic stent is coated so that the metallic stent may biodegrade after implantation in a vascular lumen and release metal hydroxide ions, and the active ingredients may have a vasodilator effect contemporarily.

Stents typically have a main body made of metal which is formed by multiple webs.

### BRIEF DESCRIPTION OF THE DRAWINGS

The figures show exemplary sections of webs of an otherwise arbitrary main body of the stent.

FIG. 1a is a web having holes of a stent main body;

FIG. 1b is a web having cavities of a stent main body;

FIG. 1c is a web having cages of a stent main body; and

FIG. 2 is a web having a “detachable” coating of a stent main body.

### DETAILED DESCRIPTION

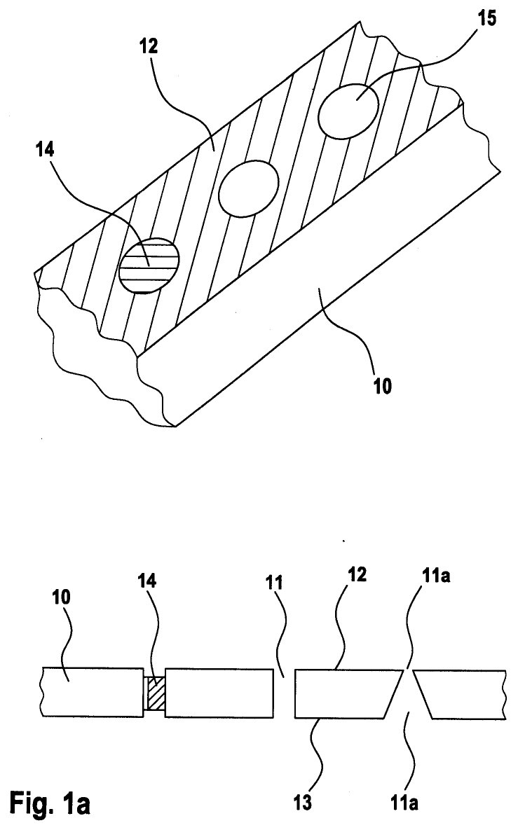

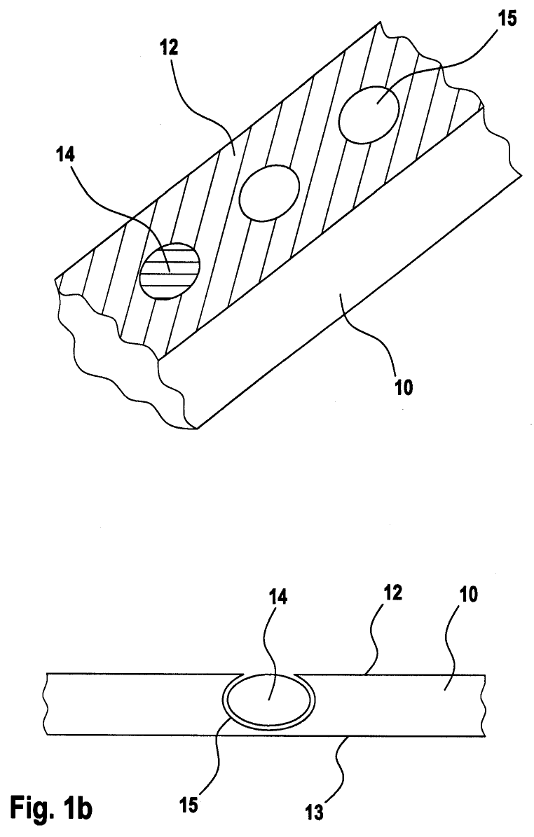

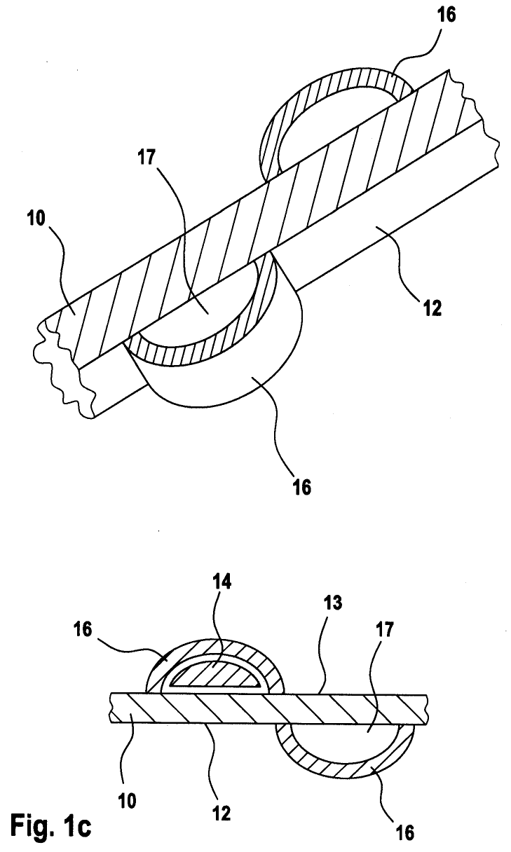

In a preferred exemplary embodiment, the suitable coating having suitable active ingredients is entirely or partially decanted into holes and/or cavities of the biodegradable stent. Such holes and/or cavities are shown in FIGS. 1a, 1b, and 1c.

FIGS. 1a, 1b, and 1c show a section of a main body 10 of a stent which is molded from the biodegradable material. The metallic material forms a filigree framework made of webs connected to one another.

FIG. 1a shows a web 10 which is drilled through in such a way that essentially cylindrical holes 11 result in the web, which lead from an external surface 12 to an internal surface 13. These holes 11 may be entirely or partially filled with a polymer coating 14 containing an active ingredient.

A stent which is suitable has already been described in International Patent Publication No. WO 2005/102222 (Conor Medsystems, Inc.) as a so-called “Conor stent” and may be used as a main body for the stent and the coating.

Alternatively or cumulatively to the holes 11, conically shaped holes 11a may also be provided, the conically shaped holes tapering toward the external surface 12, which forms the mural exterior side of the stent, so that upon dilation of the stent, the polymer coating 14 is held in the hole 11a.

Accordingly, exclusively cylindrically shaped holes 11 or conically shaped holes 11a or an arbitrary combination of cylindrically shaped holes 11 and conically shaped holes 11a may be provided in a stent.

FIG. 1b shows a web 10 which is machined in such a way that cavities 15 result in the web 10 which have an opening in the direction of the external surface 12 and are closed in the direction of the internal surface 13. These cavities 15 may be entirely or partially filled with a polymer coating 14 containing active ingredient.

Accordingly, exclusively cavities 15, but also arbitrary combinations of cavities 15 and cylindrically shaped holes 11 and/or conically shaped holes 1 la, may be provided in a stent.

Cavities may also be produced in a porous surface. Such porosities are produced by the treatment of the surface or by conversion layers. Such a method for producing a conversion layer and an implant produced according to the method is disclosed in German Patent Application No. 10 2006 060 501.2. A further method for porous coating of an object containing magnesium with a conversion layer is described in International Patent Publication No. WO 00/56950 A1.

FIG. 1c shows a web 10 which is machined in such a way that cages 16 are attached to the web 10 on the external surface 12 and/or the internal surface 13, preferably on the exterior side 12. An opening 17 is formed by these cages 16, which may be entirely or partially filled with the polymer coating 14 containing active ingredient.

Accordingly, exclusively cages 16, but also arbitrary combinations of cages 16 and cavities 15 and/or cylindrically shaped holes 11 and/or conically shaped holes 11a may be provided in a stent.

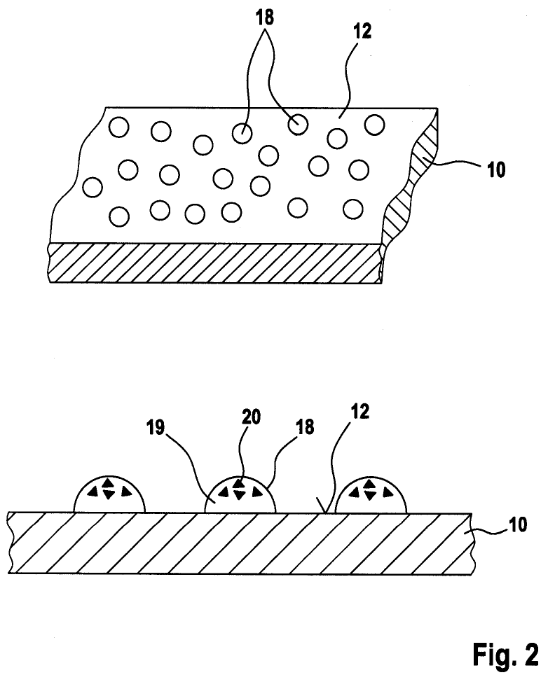

In a further preferred exemplary embodiment, the suitable coating may be coated with suitable active ingredients as a “detachable” coating. Such an exemplary embodiment is illustrated by FIG. 2.

In FIG. 2, a polymer coating containing active ingredient is applied to an external surface 12 of the web 10. As is shown, the coating area is divided into an uncoated partial area and a coated partial area.

The polymer coating containing active ingredient is implemented in the present illustrative embodiment as multiple coating islands 18 which comprise a biodegradable carrier matrix 17 and at least one active ingredient 20 (shown here as a triangle) embedded in the carrier matrix 19. The coating islands 18 are applied to the surface 12 of the main body 10 in such a way that the coated partial area, i.e., the coating islands 18, cover approximately 10-15% of the external surface 12 of the coating area.

In the present illustrative embodiment, the web 10 comprises the magnesium alloy WE 43 and the carrier matrix is high-molecular-weight poly-L-lactide (molar mass greater than 500 kD). A degradation speed of the polymer material of the carrier matrix 15 is approximately 10 to 15 times the degradation speed of the material of the main body 10.

The individual coating islands have a mean diameter of approximately 50 to 70 μm. A distance of an arbitrary point of the surface in the coated partial area to the closest uncoated partial area is thus less than 35 μm. If the coating islands are uniformly round, the distance of an arbitrary first boundary point of the surface in the coated partial area to a second boundary point which is furthest away from the first boundary point is approximately 50 to 70 μm.

The following exemplary procedure may be used for applying the coating islands 18.

The stent is pre-mounted on a balloon or catheter. A solution/extremely fine dispersion of the biodegradable polymer and at least one active ingredient is provided in a reservoir. Subsequently, droplets of a defined size are applied in selected areas of the main body via an activatable microinjection system. The solvent is withdrawn by vaporization and the coating islands of defined diameter form.

Accordingly, a “detachable” coating may be exclusively provided as the coating islands 18, but also arbitrary combinations of coating islands 18 and cages 16 and/or cavities 15 and/or cylindrically shaped holes 11 and/or conically shaped holes 11a may be provided in a stent.

The polymer coating charged with active ingredient may be bonded to the stent by anchor molecules. Anchoring using a quadrivalent silicon atom to the surface of a magnesium stent is cited here as an example, which is described, for example, in German Patent Application No. 10 2006 038 321.5.

Preferably, magnesium, iron, or tungsten alloys are suitable as the materials for biodegradable metallic stents. Magnesium alloys of the type WE, in particular, WE43 are especially preferably suitable. The latter alloy is distinguished by the presence of rare earth elements and yttrium. The cited materials may be easily processed, have low material costs, and are especially suitable for the production of stents because of the relatively rapid degradation and the more favorable elastic behavior than polymers (lower recoil of the stent). In addition, a positive physiological effect of the degradation products on the healing process has been established for at least a part of the alloys. Furthermore, it has been shown that magnesium stents produced from WE43 do not generate interfering magnetic resonance artifacts, as are known, for example, from medical stainless steel (316A) and, therefore, treatment success may be tracked using detection devices based on magnetic resonance. The biodegradable metal alloys made of the elements magnesium, iron, or tungsten preferably contain the cited elements in a proportion of at least 50 weight-percent, in particular, at least 70 weight-percent, especially preferably at least 90 weight-percent, of the alloy.

Biodegradable metallic stents may be produced and particularly coated using methods of the part.

The above-mentioned preferred exemplary embodiments may each be applied to the following embodiments of the present disclosure.

A second aspect of the present disclosure relates to a method for improving the stability of a biodegradable metallic stent having the following steps:
providing a biodegradable metallic stent,
providing one or more vasodilator active ingredients selected from the group consisting of calcium channel blockers, nitrates, rho-kinase inhibitors, endothelin receptor antagonists, serotonin antagonists, adrenoreceptor antagonists, potassium channel openers, angiotensin conversion enzyme (ACE) inhibitors, musculotropic and/or neurotropic-musculotropic spasmolytics, and the like, vasodilators having unknown action mechanisms, and the active ingredients which regulate down spasmogenically active messenger agents, and
coating the biodegradable metallic stent with the vasodilator active ingredient(s) in a suitable matrix,
the biodegradable metallic stent being coated in such a way that the vasodilator active ingredient(s) has/have a vasodilator effect in the area of the stent implantation contemporarily to the biodegradation of the stent after implantation.

A third aspect of the present disclosure relates to a method for producing a biodegradable metallic stent having the following steps:
providing a biodegradable metallic stent,
providing one or more vasodilator active ingredients selected from the group consisting of calcium channel blockers, nitrates, rho-kinase inhibitors, endothelin receptor antagonists, serotonin antagonists, adrenoreceptor antagonists, potassium channel openers, angiotensin conversion enzyme (ACE) inhibitors, musculotropic and/or neurotropic-musculotropic spasmolytics, and the like, vasodilators having unknown action mechanisms, and the active ingredients which regulate down spasmogenically active messenger agents, and
coating the biodegradable metallic stent with the vasodilator active ingredient(s) in a suitable matrix,
the biodegradable metallic stent being coated in such a way that the vasodilator active ingredient(s) has/have a vasodilator effect in the area of the stent implantation contemporarily to the biodegradation of the stent after implantation.

A fourth aspect of the present disclosure relates to the use of one or more vasodilator active ingredients for improving the stability of a biodegradable metallic stent, wherein a biodegradable metallic stent is coated with one or more vasodilator active ingredients in a suitable matrix in such a way that the vasodilator active ingredient(s) has/have a vasodilator effect in the area of the stent implantation contemporarily to the biodegradation of the stent after implantation. The present disclosure also provides a method for producing a biodegradable metallic stent using one or more vasodilator active ingredients produced by a method provided in the present disclosure.

A fifth aspect of the present disclosure relates to a method for therapeutic or prophylactic treatment of humans or animals having the following steps:
providing a biodegradable metallic stent,
providing one or more vasodilator active ingredients selected from the group consisting of calcium channel blockers, nitrates, rho-kinase inhibitors, endothelin receptor antagonists, serotonin antagonists, adrenoreceptor antagonists, potassium channel openers, angiotensin conversion enzyme (ACE) inhibitors, musculotropic and/or neurotropic-musculotropic spasmolytics, and the like, vasodilators having unknown action mechanisms, and the active ingredients which regulate down spasmogenically active messenger agents,
implanting the biodegradable metallic stent in a vessel, and
administering the vasodilator active ingredient(s) in a quantity which is sufficient to prevent or reduce vascular spasms in the area of the stent implantation.

The suitable vasodilator agents may additionally be provided together with further pharmaceutically acceptable auxiliary materials, carriers, and solutions.

Such stents are implanted according to the typical methods. For this purpose, stents are inserted into the vessel in a compressed state and then expanded at the location to be treated and pressed against the vascular wall. This expansion may be performed with the aid of a balloon catheter, for example. Alternatively, self-expanding stents are also known. These are produced from a memory metal, such as nitinol, for example.

The administration, preferably a peroral administration of the vasodilator active ingredient(s), is begun contemporarily, preferably shortly before stent implantation up to simultaneously with the stent implantation, and continued in a quantity which is sufficient to prevent or reduce a vascular spasm in the area of the stent implantation as the stent biodegrades, preferably while the quantity of hydroxide ions released by the biodegradation is sufficient to trigger or mediate a vascular spasm in the area of the stent implantation, more preferably within a period of time of at least 4 months, particularly preferably within a period of time of at least 3 to 8 weeks after stent implantation.

A further aspect of the present disclosure relates to a method for therapeutic or prophylactic treatment of humans or animals having the following steps:
providing a biodegradable metallic stent according to the present disclosure, and
implanting the biodegradable metallic stent in a vessel.

In a preferred exemplary embodiment, the above-mentioned therapeutic or prophylactic method is used for improving the stability of biodegradable metallic stents.

A stent according to the present disclosure is preferably implanted in arteries, more preferably in large arteries.

The present disclosure was described in detail form via the preferred exemplary embodiments. Such embodiments solely represent examples, however, and may also be altered by those skilled in the art in equivalent ways, if necessary, which are also included by the present disclosure.

All patents, patent applications and publications referenced herein are incorporated by reference herein in their entirety.

What is claimed is:

1. A biodegradable metallic stent, comprising:
(a) a coating comprising one or more vasodilator active ingredients selected from the group consisting of calcium channel blockers, nitrovasodilators, rho-kinase inhibitors, endothelin receptor antagonists, serotonin antagonists, adrenoreceptor antagonists, potassium channel openers, angiotensin conversion enzyme (ACE) inhibitors, musculotropic and neurotropic-musculotropic spasmolytics, and vasodilators having unknown action mechanisms, the active ingredient or the active ingredients of which regulate down spasmogenically active messenger agents, phosphodiesterase-5 (PDE-5) inhibitors, TRP inhibitors or activators, the active ingredients which activate or inhibit TRP channels and
(b) a matrix associated with the coating such that the vasodilator active ingredient has a vasodilator effect in the area of the stent implantation contemporarily to the biodegradation of the stent after implantation.

2. The stent of claim 1, wherein the elution time of the one or more vasodilator active ingredients from the matrix [TE(W)] corresponds to the degradation time of the stent [TD(S)].

3. The stent of claim 1, wherein the elution time of the active ingredient from the matrix [TE(W)] corresponds to the degradation time of the active ingredient depot of the matrix on the stent [TD(D)], and
wherein [TD(D)] and [TE(W)] are each less than the degradation time of the stent [TD(S)], and
wherein [TD(S)] is less than or equal to the elution time of the active ingredient from the vascular wall [TE(G)].

4. The stent of claim 1, wherein the degradation time of the stent [TD(S)] is less than the elution time of the active ingredient from the vascular wall [TE(G)] or is less than the elution time of the active ingredient from the matrix of the stent [TE(W)], and wherein [TE(G)] corresponds to [TE(W)].

5. The stent of claim 1, wherein the coating comprises a plurality of the vasodilator active ingredients and the vasodilator active ingredients elute independently of one another.

6. The stent of claim 1, wherein the matrix further comprises one or more active ingredients from the group consisting of anti-inflammatory active ingredients, antiproliferative active ingredients, antisense nucleotides, biphosphonates, antibodies, and progenitor cells in the matrix.

7. The stent of claim 1, wherein the stent further comprises at least one cavity and the coating is decanted into the cavities.

8. A method for improving the stability of a biodegradable metallic stent, comprising the steps of:
(a) providing a biodegradable metallic stent;
(b) providing one or more vasodilator active ingredients selected from the group consisting of calcium channel blockers, nitrovasodilators, rho-kinase inhibitors, endothelin receptor antagonists, serotonin antagonists, adrenoreceptor antagonists, potassium channel openers, angiotensin conversion enzyme (ACE) inhibitors, musculotropic and neurotropic-musculotropic spasmolytics, and vasodilators having unknown action mechanisms, the active ingredients of which regulate down spasmogenically active messenger agents, phosphodiesterase-5 (PDE-5) inhibitors, TRP inhibitors or activators, the active ingredients which activate or inhibit TRP channels; and
(c) coating the biodegradable metallic stent with the vasodilator active ingredient in a suitable matrix,
wherein the biodegradable metallic stent is coated so that the vasodilator active ingredient has a vasodilator effect in the area of the stent implantation contemporarily to the biodegradation of the stent after implantation.

9. A method for producing a biodegradable metallic stent, comprising:
(a) providing a biodegradable metallic stent,
(b) providing one or more vasodilator active ingredients selected from the group consisting of calcium channel blockers, nitrates, rho-kinase inhibitors, endothelin receptor antagonists, serotonin antagonists, adrenoreceptor antagonists, potassium channel openers, angiotensin conversion enzyme (ACE) inhibitors, musculotropic and neurotropic-musculotropic spasmolytics, and vasodilators having unknown action mechanisms, the active ingredients of which regulate down spasmogenically active messenger agents, phosphodiesterase-5 (PDE-5) inhibitors, TRP inhibitors or activators, and the active ingredients which activate or inhibit TRP channels; and
(c) coating the biodegradable metallic stent with the one or more vasodilator active ingredient in a suitable matrix,
wherein the biodegradable metallic stent is coated in such a way that the vasodilator active ingredient has a vasodilator effect in the area of the stent implantation contemporarily to the biodegradation of the stent after implantation.

10. A method for improving the stability of a biodegradable metallic stent, comprising:
coating a biodegradable metallic stent with one or more vasodilator active ingredients in a suitable matrix such that the one or more vasodilator active ingredients have a vasodilator effect in the area of the stent implantation contemporarily to the biodegradation of the stent after implantation.

11. The stent of claim 1, wherein the cavities are tapered in diameter toward the mural side.
